(19) United States
(12) Patent Application Publication
(10) Pub. No.: US 2020/0015769 A1
(43) Pub. Date: Jan. 16, 2020

(54) X-RAY COMPUTED TOMOGRAPHY APPARATUS AND CORRECTION METHOD

(71) Applicant: Hitachi, Ltd., Tokyo (JP)

(72) Inventors: Shinichi KOJIMA, Tokyo (JP); Fumito WATANABE, Tokyo (JP); Kazuma YOKOI, Tokyo (JP); Fuyuhiko TERAMOTO, Tokyo (JP)

(21) Appl. No.: 16/439,748

(22) Filed: Jun. 13, 2019

(30) Foreign Application Priority Data
Jul. 10, 2018 (JP) 2018-130538

(51) Int. Cl. A61B 6/00

(52) U.S. Cl. CPC A61B 6/5282; A61B 6/4291

(57) ABSTRACT

To provide an X-ray CT apparatus capable of correcting a difference of scattered radiation quantities contained in the projection data even in a case where a plurality of detection elements are disposed between collimator plates. The X-ray CT apparatus includes: an X-ray irradiation section for X-ray radiation; an X-ray detector including a plurality of detection elements for detecting the X-ray; a plurality of collimator plates disposed between the X-ray irradiation section and the X-ray detector so as to reduce scattered radiation; a reconstruction portion for reconstructing a tomographic image by using projection data generated based on an output from the X-ray detector; and a correction portion for correcting the projection data by using different correction functions according to the positions of the plural detection elements disposed between the collimator plates.

### CLAIM OF PRIORITY

The present application claims priority from Japanese Patent Application JP 2018-130538 filed on Jul. 10, 2018, the content of which are hereby incorporated by references into this application.

### TECHNOLOGICAL FIELD

The present invention relates to an X-ray computed tomography apparatus, and more particularly to a technique for correcting projection data acquired by the X-ray computed tomography apparatus.

### BACKGROUND

An X-ray computed tomography apparatus acquires pieces of projection data of an object at various projection angles and reconstructs a tomographic image based on plural pieces of projection data. The X-ray computed tomography apparatuses are widely used in fields of inspection apparatuses for industrial or security purposes and medical diagnostic imaging units. As for the medical diagnostic imaging units, there is a strong need for improvement in the spatial resolution of tomographic images. For example, images of the inside of a stent inserted through narrowed blood vessels are required of such a high spatial resolution as to permit determination of the presence of recurrent stenosis or the follow-up observation of the characteristics of plaques.

The improvement of spatial resolution requires the miniaturization of detection elements for X-ray detection. An increasing number of collimator plates tends to be added in conjunction with the progress of miniaturization of the detection elements. The collimator plate is a slit plate disposed at a stage preceding the detection element in order to reduce scattered radiation incident on the detection element. Since the collimator plate has a finite depth, an excessive addition of the collimator plates results in the reduction of quantity of X-ray radiation incident on the detection elements and the deterioration of S/N ratio. There is known a technique of arranging a plurality of detection elements between the collimator plates so as to suppress the S/N ratio deterioration. With this technique, however, detection data is degraded in accuracy because of the X-ray beams incident on the adjoining detection elements.

Japanese Patent Application Laid-Open No. 2010-220880 discloses a radiation detector which is directed to suppress the degradation of detection data accuracy and to increase productivity. This radiation detector includes a region where a plurality of detection elements are disposed between the collimator plates and a region where a single detection element is disposed between the collimator plates.

### SUMMARY OF THE INVENTION

However, Japanese Patent Application Laid-Open No. 2010-220880 does not give consideration to a difference in incident direction of scattered radiation unremoved by the collimator plates on the individual detection elements in the region where the plural detection elements are disposed between the collimator plates. The direction of the gaze to between the collimator plates from the detection element varies depending upon the location of the detection element between the collimator plates. Hence, the quantity of scattered radiation incident on the detection element differs depending upon the position of the detection element between the collimator plates. The tomographic image reconstructed using projection data containing the scattered radiation quantity differences suffers from the occurrence of artifact that interferes with diagnosis.

In this connection, an object of the present invention is to provide an X-ray CT apparatus which is capable of correcting the scattered radiation quantity differences contained in the projection data even when a plurality of detection elements are disposed between collimator plates.

For achieving the above object, the present invention is characterized by correcting the projection data by use of different correction functions according to the positions of the detection elements disposed between the collimator plates.

According to an aspect of the present invention, an X-ray CT apparatus includes: an X-ray irradiation section for X-ray radiation; an X-ray detector including a plurality of detection elements for detecting the X-ray; a plurality of collimator plates disposed between the X-ray irradiation section and the X-ray detector so as to reduce scattered radiation; a reconstruction portion for reconstructing a tomographic image by using projection data generated based on an output from the X-ray detector; and a correction portion for correcting the projection data by using different correction functions according to the positions of the plural detection elements disposed between the collimator plates.

According to another aspect of the present invention, a correction method for correcting projection data generated by an X-ray CT apparatus including a plurality of detection elements disposed between a plurality of collimator plates, includes: a step of acquiring the projection data; and a step of correcting the projection data by using different correction functions according to positions of the detection elements disposed between the collimator plates.

The present invention can provide the X-ray CT apparatus capable of correcting the scattered radiation quantity differences contained in the projection data even in the case where a plurality of detection elements are disposed between the collimator plates.

### BRIEF DESCRIPTION OF THE DRAWINGS

FIG. 1 is a schematic diagram showing a configuration of an X-ray CT apparatus according to a first embodiment of the present invention;

FIGS. 2A and 2B are diagrams illustrating an X-ray detector according to the first embodiment hereof, and a gaze angle and gaze direction of a detection element between collimator plates;

FIGS. 3A and 3B are graphs showing an example of direct radiation quantity and scattered radiation quantity of each detection element as determined by Monte Carlo simulation;

FIGS. 4A and 4B are diagrams illustrating scattered radiation quantity differences caused by the difference in the direction of the gaze to between the collimator plates from the detection element;

FIG. 5 is a flow chart showing the steps of a process for creating a correction function according to the first embodiment hereof;

FIG. 6 is a chart showing an example of correction function format according to the first embodiment hereof;

FIG. 7 is a flow chart showing the steps of scanning process including a correction process according to the first embodiment hereof;

FIG. 8 is a diagram showing an example where three detection elements are disposed between the collimator plates;

FIG. 9 is a diagram illustrating a case where a correction process according to a second embodiment hereof is performed in an arrangement where three detection elements are disposed between the collimator plates;

FIG. 10 is a flow chart showing the steps of the correction process according to the second embodiment hereof;

FIG. 11 is a diagram illustrating a case where the correction process of the second embodiment hereof is performed in an arrangement where four detection elements are disposed between the collimator plates; and

FIG. 12 is a diagram illustrating a case where the correction process of the second embodiment hereof is performed in an arrangement where five detection elements are disposed between the collimator plates.

### DETAILED DESCRIPTION OF EMBODIMENTS

An X-ray CT apparatus according to the embodiment of the present invention will hereinbelow be described with reference to the accompanying drawings. x, y, and z coordinates will be added as needed to indicate directions in respective drawings.

### First Embodiment

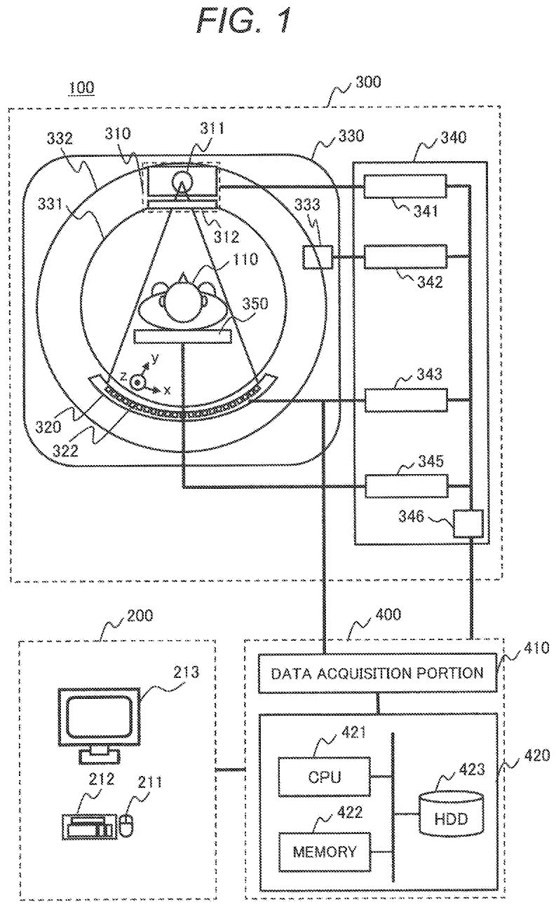

A schematic configuration of an X-ray CT apparatus 100 according to a first embodiment of the present invention is described with reference to FIG. 1. The X-ray CT apparatus 100 includes an input/output section 200, a scanning section 300, and an image generation section 400.

The input/output section 200 includes a mouse 211, a keyboard 212, and a monitor 213. The mouse 211 and keyboard 212 are input devices used by an operator to input scanning conditions and the like. The monitor 213 is a display device which outputs the inputted scanning conditions and the like. The monitor is also used as an input device if it is equipped with a touch panel function.

The scanning section 300 includes an X-ray generator 310, an X-ray detector 320, a gantry 330, a table 350, and a scanning control unit 340 in order to acquire projection data of an object 110 at various projection angles.

The X-ray generator 310 includes an X-ray tube 311 and an X-ray irradiation width controller 312. The X-ray tube 311 is a device for irradiating the object 110 with X-ray beam. The X-ray irradiation width controller 312 is a device for adjusting a z-direction length as a width of the X-ray beam illuminated onto the object 110.

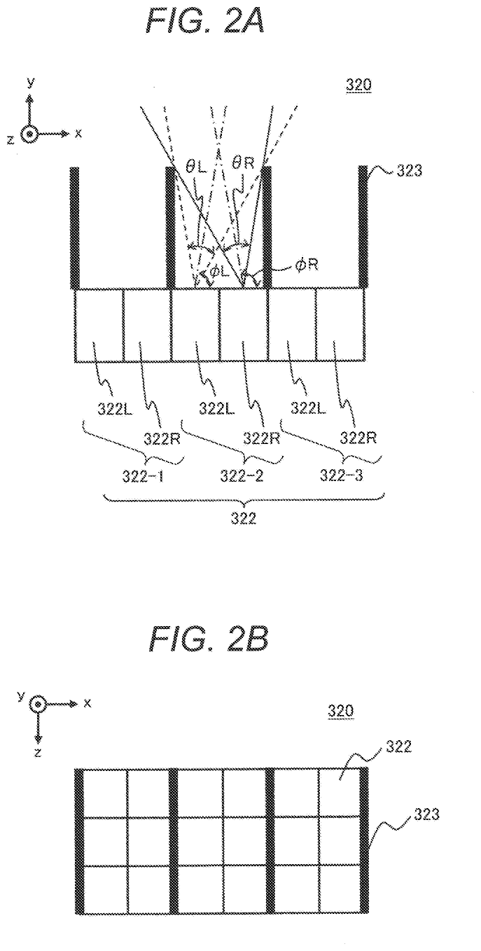

The X-ray detector 320 is a device for detecting a direct radiation as the X-ray beam transmitted through the object 110 without being scattered by the object 110. The detector 320 includes a plurality of detection elements 322. Two thousand detection elements 322 are arranged at the same distance, such as 1000 mm, from an X-ray generation point of the X-ray tube 311. The details of the X-ray detector 320 will be described hereinafter with reference to FIGS. 2A and 2B.

The gantry 330 is centrally formed with a circular aperture 331 to accommodate a table 350 to carry the object 110 thereon. The aperture 331 has a diameter of 700 mm, for example. A rotating plate 332 equipped with the X-ray generator 310 and the X-ray detector 320, and a rotation driver 333 for rotating the rotating plate 332 are disposed in the gantry 330. The table 350 is movable in a z-direction for positional adjustment of the object 110 with respect to the gantry 330.

The scanning control unit 340 includes an X-ray controller 341, a gantry controller 342, a detector controller 34, a table controller 345, and an integrated controller 346. The X-ray controller 341 controls voltage and the like applied to the X-ray tube 311. The gantry controller 342 controls the rotary drive of the rotating plate 332. The detector controller 343 controls the X-ray detection by the X-ray detector 320. The table controller 345 controls the movement of the table 350. The integrated controller 346 controls the flow of operations of the X-ray controller 341, the gantry controller 342, the detector controller 343, and the table controller 345 based on the scanning conditions inputted by the input/output section 200. For instance, the rotating plate 332 is rotated at 1.0 second/revolution while the X-ray beam is detected at 0.4 degree/revolution.

The image generation section 400 includes a data acquisition portion 410 and a data processor 420. The data acquisition portion 410 converts a detection result supplied from the X-ray detector 320 to a digital signal. The data processor 420 includes a central processing unit (CPU) 421, a memory 422, and a hard disk drive (HDD) 423. The central processing unit 421 and the memory 422 perform a correction process, a tomographic image reconstruction process and the like by expanding and starting predetermined programs. Namely, the data processor 420 functions as a correction portion performing the correction process and a reconstruction portion performing the reconstruction process. The HDD 423 stores, inputs, and outputs data. The constructed tomographic images and the like may be displayed on the monitor 213 of the input/output section 200 or, may be displayed on a display device connected via a network. The input/output section 200 and the image generation section 400 need not be installed in the X-ray CT apparatus 100 and the operations thereof may be implemented by means of, for example, another device connected via a network.

The X-ray detector 320 of the embodiment is described with reference to FIGS. 2A and 2B. FIGS. 2A and 2B show a part of the X-ray detector 320. FIG. 2A shows an x-y cross section of the detector while FIG. 2B shows an x-z cross section thereof. The X-ray detector 320 of the embodiment is configured to include a plurality of detection elements 322 and a plurality of collimator plates 323.

The detection element 322 is a device for detecting the X-ray beam and outputs an electric signal corresponding to a quantity of the X-ray beam incident on one element. The detection elements 322 are arranged on an x-y plane and has a size of 0.5 mm square, for example. The detection element 322 may be an indirect detection element essentially including a combination of a scintillator element and a photodiode element or may also be a semiconductor detection element represented by CdTe. In the indirect detection element, the incident X-ray beam cause the scintillator element to emit fluorescent light which is converted to the electric signal by the photodiode element.

The collimator plate 323 is a slit plate disposed at stage preceding the detection element 322 in order to reduce the scattered radiation from the object 110 and the like. The collimator plate is a plate material made of a heavy metal such as molybdenum and tungsten. The collimator plate 323 is disposed at a boundary between the adjoining detection elements 322 and in parallel to the direct radiation beams transmitted through the object 110 so that almost all of the direct radiation beams become incident on the detection element 322 while most of the scattered radiation is absorbed by the collimator plate 323. However, the collimator plate has the finite depth. Therefore, as an increasing number of collimator plates 323 is added in conjunction with the miniaturization of the detection elements 322, the quantity of the direct radiation incident on the detection elements 322 decreases. In order to suppress the decrease of the direct radiation, the number of collimator plates 323 is reduced while a plurality of detection elements 322 are disposed between the collimator plates 23.

According to the embodiment, two detection elements 322L and 322R are disposed between the collimator plates 323. FIG. 2A shows three detection element groups 322-1, 322-2, 322-3, each of which includes the detection element 322L and the detection element 322R. An angle θL formed between two dotted lines respectively connecting the center of the detection element 322L with an upper end of the collimator plate 323 is defined as a gaze angle of the detection element 322L between the collimator plates 323. Further, angle θR formed between two solid lines respectively connecting the center of the detection element 322R with an upper end of the collimator plate 323 is defined as a gaze angle of the detection element 322R between the collimator plates 323.

Although the gaze angle θL of the detection element 322L and the gaze angle θR of the detection element 322R have the same value, the directions of the gaze to between the collimator plates 323 from the respective detection elements 322 differ from each other. That is, angles φL and φR formed between respective dot-dash lines as bisectors of the angles φL and φR and the top surface of the detection element 322 have different values. The inventors have performed the Monte Carlo simulation and found that the different directions φL and φR of the gaze to between the collimator plates 323 from the respective detection elements 322 lead to the different quantities of scattered radiation incident on the detection element 322L and the detection element 322R.

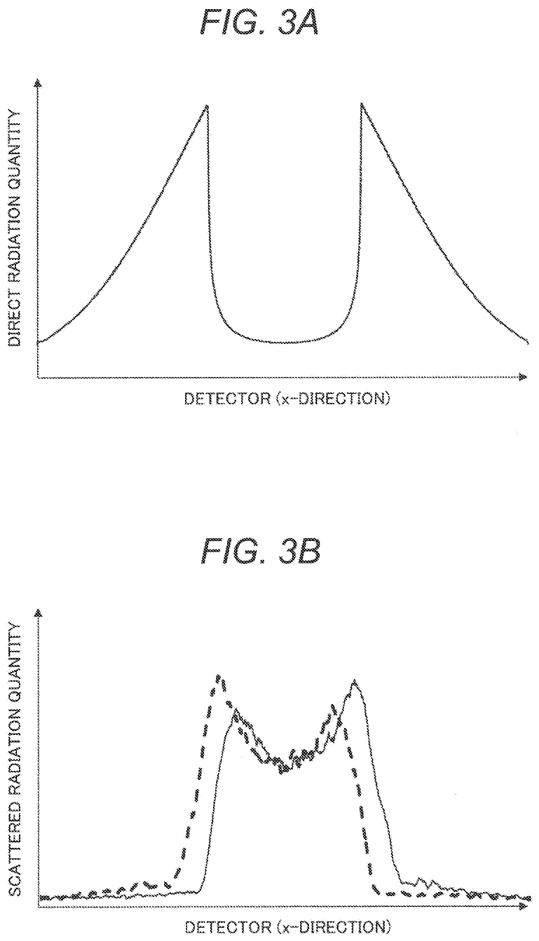

Now referring to FIGS. 3A and 3B, description is made on an example of the results of the Monte Carlo simulation where water phantom having a diameter of 150 cm is irradiated with X-ray in an arrangement where two detection elements 322L, 322R are disposed between the collimator plates 323. FIG. 3A shows an x-direction distribution of the quantity of direct radiation incident on each detection element 322 without being scattered by the water phantom. FIG. 3B shows an x-direction distribution of the quantity of radiation scattered by the water phantom and incident on each detection element 322 without being absorbed by the collimator plates 323. In FIGS. 3A and 3B, the dot line represents the quantity of X-ray radiation incident on the detection element 322L while the solid line represents the quantity of X-ray radiation incident on the detection element 322R.

In FIG. 3A, the dot line and the solid line substantially overlap with each other, indicating that there is little difference between the quantities of direct radiations incident on the detection element 322L and the detection element 322R and that the X-ray is attenuated in a range where the water phantom as the object 110 is placed. In FIG. 3B, on the other hand, the dot line and the solid line deviate from each other in vicinities of two peaks of the distribution of direct radiation quantity which correspond to peripheral areas of the water phantom, indicating that the quantity of scattered radiation incident on the detection element 322L differs from the quantity of scattered radiation incident on the detection element 322R. The X-ray radiation detected by the X-ray detector 320 of the X-ray CT apparatus 100 is the sum of the direct radiation quantity shown in FIG. 3A and the scattered radiation quantity shown in FIG. 3B. The difference in the scattered radiation quantity shown in FIG. 3B becomes a cause of the artifact in the tomographic images. Hence, the difference of scattered radiation quantity shown in FIG. 3B must be corrected before tomographic image reconstruction.

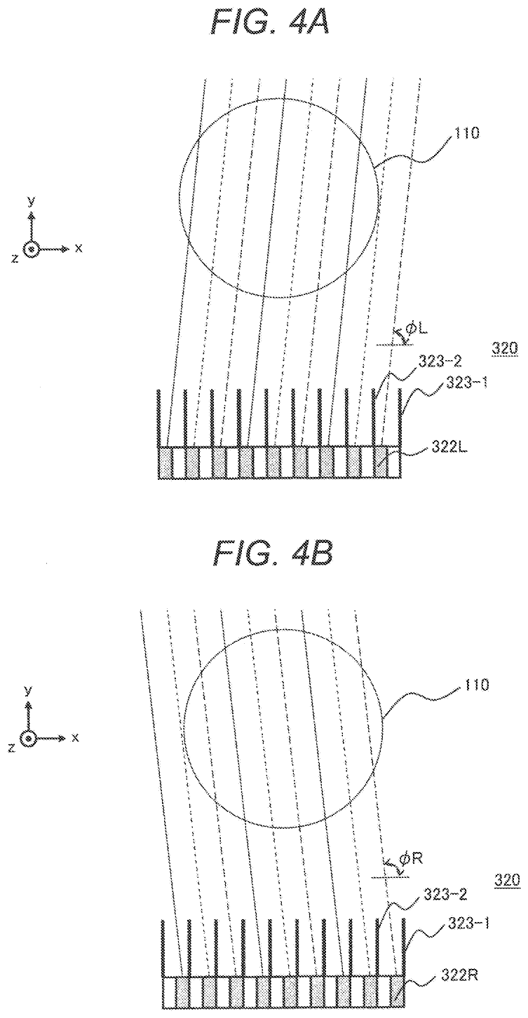

Now referring to FIGS. 4A and 4B, description is made on the difference of scattered radiation quantity resulting from the difference of the directions φL and φR of the gaze to between the collimator plates 323 from the respective detection elements 322. FIG. 4A shows a path of scattered radiation incident on the detection elements 322L in the direction φL of the gaze thereto. FIG. 4B shows a path of scattered radiation incident on the detection elements 322R in the direction φR of the gaze thereto. It is found from comparison between FIG. 4A and FIG. 4B that there is a case where on the scattered radiation paths to the detection element 322L and the detection element 322R between the same collimator plates 323, the object 110 exists on the path to one detection element but does not exist on the path to the other detection element. For example, the object 110 exists on a path to the detection element 322R between a collimator plate 323-1 and a collimator plate 323-2 but does not exist on a path to the counterpart detection element 322L. That is, even on the detection elements 322 between the same collimator plates 323, the scattered radiation paths to these elements differ because of the different directions φL and φR of the gaze thereto between the collimator plates 323, resulting in the difference in the scattered radiation quantity. The difference of directions φL and φR of the gaze to between the collimator plates 323 depends upon whether the detection element 322 is on the left side or the right side of space between the collimator plates 323.

According to the embodiment, therefore, a correction function for the detection element 322L and a correction function for the detection element 322R are previously created in a case where the two detection elements 322L, 322R are disposed between the collimator plates 323. Then, the projection data acquired by the X-ray CT apparatus 100 is corrected with the previously created correction functions so as to reconstruct the tomographic image from the corrected projection data.

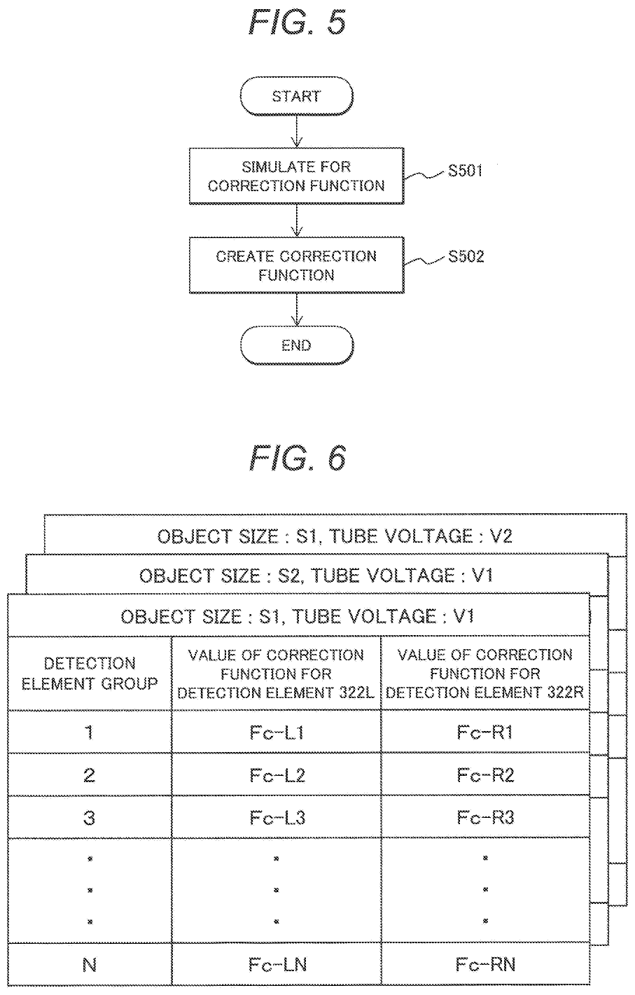

A flow of steps of creating the correction function for the detection element 322L and the correction function for the detection element 322R is described with reference to FIG. 5.

### (S501)

The data processor 420 performs the Monte Carlo simulation for the creation of correction function. Cylindrical water phantoms or polyethylene phantoms having different diameters are used as a virtual object 110 for use in simulation. Values of real machines are applied to a geometric structure of the X-ray CT apparatus 100 including the X-ray detector 320 and the X-ray tube 311.

In the Monte Carlo simulation in this step, the direct radiation incident and scattered radiation incident on each detection element 322L and each detection element 322R are calculated as illustrated in FIGS. 3A and 3B while changing the size of the object 110 and the tube voltage of the X-ray tube 311. This step is not limitedly performed by the data processor 420 but may also be performed using an arithmetic device external to the X-ray CT apparatus 100.

### (S502)

Using the results obtained in the step S501, the data processor 420 calculates a ratio between the direct radiation quantity and the scattered radiation quantity for each detection element 322L and each detection element 322R. In conjunction with the increase or decrease of the direct radiation quantity, the scattered radiation quantity varies in proportion to the direct radiation quantity. Therefore, the previous calculation of the ratio between the direct radiation quantity and the scattered radiation quantity facilitates a response to the increase or decrease of tube current of the X-ray tube 311, for example.

In the Monte Carlo simulation, the calculation results fluctuate depending upon the photon number of the emitted X-ray beam. Therefore, fitting using, for example, a quadric function may be applied to the ratio between the direct radiation quantity and the scattered radiation quantity so as to facilitate the use of the correction function. The quadric function resulting from the fitting is referred to as characteristic curve. The characteristic curve is determined each time the size of the object 110 or the tube voltage of the X-ray tube 311 is changed. The resultant characteristic curve is stored, as the correction function, in the HDD 423 or the storage device external to the X-ray CT apparatus 100.

FIG. 6 shows an example of the format of the correction functions stored in the storage device. The correction functions are stored as a table including detection element group Numbers and values of the correction functions for the detection elements 322L and detection elements 322R. The table is created each time the object size and the tube voltage are changed. The detection element group Number is defined to mean a number assigned to a pair of the detection elements 322L and 322R. One detection element group number is in correspondence to the detection elements 322L and 322R.

The correction function for the detection element 322L and the correction function for the detection element 322R are created by the above-described process flow. The process flow of FIG. 5 is performed during the manufacture of the X-ray CT apparatus 100 or at the time of replacement of the X-ray tubes 311 or the X-ray detectors 320.

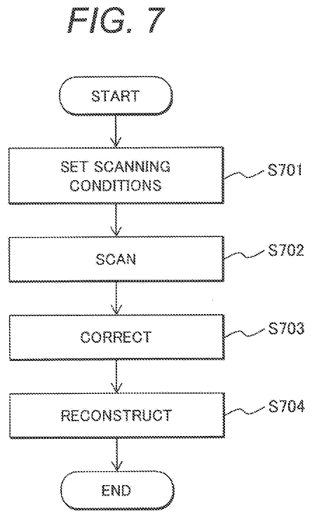

Now referring to FIG. 7, description is made on a flow of scanning process including a correction process using the correction functions for the detection element 322L and for the detection element 322R.

### (S701)

The scanning conditions for the X-ray CT apparatus 100 are set by the operator through the input/output section 200. Specifically, the operator manipulates the mouse 211, the keyboard 212 and the like while watching an input screen on the monitor 213, so as to set tube current and tube voltage of the X-ray tube 311, an open width of the X-ray irradiation width controller 312, a scanning range of the object 110, a rotational rate of the rotating plate 332 and the like. The apparatus may also be adapted to allow the operator to retrieve and set some of the previously registered scanning conditions as required so as to negate the need for the operator to make settings each time the scanning work is performed.

### (S702)

Upon receiving a scanning start command from the operator, the integrated controller 346 performs the scanning work based on the scanning conditions set in the step S701. Specific steps of the procedure are described as below.

First, after the object 110 is placed on the table 350, the integrated controller 346 instructs the table controller 345 to move the table 350 so as to place the object 110 at a scanning position in the gantry 330. When the object 110 is positioned at place, the integrated controller 346 instructs the gantry controller 342 to drive the rotation driver 333 so as to start the rotation of the rotating plate 332.

When the rotating plate 332 reaches a constant speed rotation, the integrated controller 346 instructs the X-ray controller 341 to perform the X-ray radiation from the X-ray tube 311 and also instructs the detector controller 343 to perform the X-ray detection by the X-ray detector 320 and to transmit the detection data to the data acquisition portion 410. The detection data transmitted to the data acquisition portion 410 is stored in the HDD 423. Further, the integrated controller 346 instructs the table controller 345 to move the table 350 so as to scan the object 110 in the scanning range set in the step S701.

When the scanning in the scanning range is completed, the integrated controller 346 stops the X-ray radiation from the X-ray tube 311, the X-ray detection by the X-ray detector 320, and the transmission of the detection data to the data acquisition portion and returns the table 350 to a predetermined position.

### (S703)

The data processor 420 obtains corrected projection data by performing the correction processing, along with logarithmic conversion processing and Air correction processing, on the detection data acquired in the step S702 by using the correction functions created in the step S502. The logarithmic conversion processing and the Air correction processing are the same as those known in the art and hence, the description thereof is dispensed with. Specific steps of the correction processing using the correction functions are described as below.

First, the data processor 420 separates the detection data into data acquired by the detection element 322L and data acquired by the detection element 322R. Since each of the data pieces contains not only the direct radiation quantity but also the scattered radiation quantity, the scattered radiation quantity is removed by the following processing.

Next, the data processor 420 retrieves the correction functions corresponding to the scanning conditions from the storage device such as the HDD 423. Namely, the data processor retrieves a table corresponding to the scanning conditions from among the plural tables as shown in FIG. 6. In a case where a correction function matching the scanning condition is not stored in the storage device such as the HDD 423, a correction function for a scanning condition closest to the relevant scanning condition or a function created by weighting interpolation of a plurality of correction functions may be used. In a case where correction functions for virtual objects 110 having sizes of 100 mm, 200 mm, 300 mm, 400 mm are stored, for example, a correction function for the object having a size of 200 mm may be used to scan an object 110 having a size of 220 mm. Otherwise, a function created by weighting interpolation of the correction functions for the objects having a diameter of 200 mm and a diameter of 300 mm may be used to scan the object 110 having the size of 220 mm. The correction processing is increased in accuracy by using the correction function matching the scanning conditions.

Finally, the data processor 420 removes the scattered radiation quantity from the data acquired by the detection element 322L and from the data acquired by the detection element 322R by using the resultant correction functions. Data Dm acquired by the detection element 322 is equivalent to “the direct radiation quantity+the scattered radiation quantity” and a correction function Fc is equivalent to “the scattered radiation quantity/direct radiation quantity”. Therefore, it suffices to calculate Dm/(1+Fc) to remove the scattered radiation quantity and determine the direct radiation quantity.

The corrected projection data is generated by performing the correction processing using the above-described correction function along with the logarithmic conversion processing and Air correction processing.

### (S704)

The data processor 420 reconstructs the tomographic image by using the corrected projection data obtained in the step S703. The Feldkamp method or a successive approximate reconstruction method is used for the reconstruction of the tomographic image. The resultant tomographic image is displayed on the monitor 213 or the like and used for diagnosis of the object 110.

In conformity to the above-described process flow, the tomographic image is reconstructed from the projection data corrected with the correction function created according to the embodiment. Therefore, the occurrence of artifact can be suppressed even in the case where two detection elements 322L and 322R are disposed between the collimator plates 323. Further, the number of collimator plates 323 is decreased so that the direct radiation reduction by the collimator plates 323 can be suppressed. According to the embodiment, ineffective exposure can be reduced more than before.

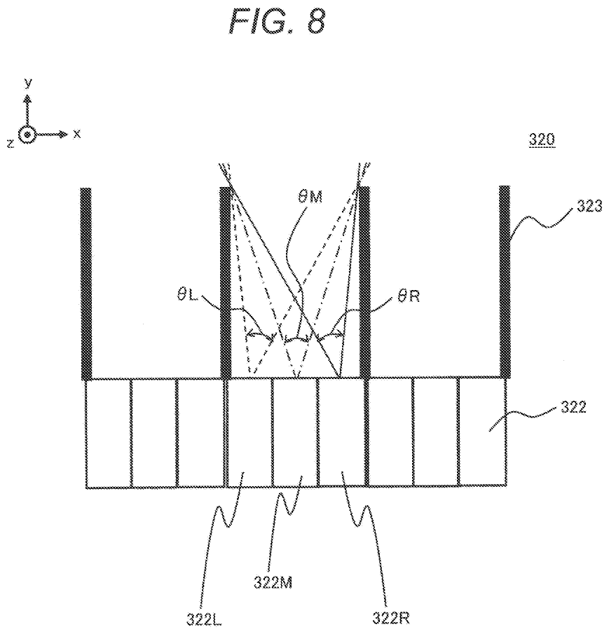

It is noted that the number of the detection elements 322 disposed between the collimator plates 323 is not limited to two but three or more detection elements can be disposed therebetween. FIG. 8 shows an example where three detection elements 322 are disposed between the collimator plates 323. It is assumed that a left-hand element is referred to as “detection element 322L”, a central element is referred to as “detection element 322M”, and a right-hand element is referred to as “detection element 322R”. Between the collimator plates 323, “θL” denotes a gaze angle of the detection element 322L, “θL” denotes a gaze angle of the central detection element 322M, and “θR” denotes a gaze angle of the detection element 322R.

The gaze angle θL and the gaze angle θR have the same value just as in the case of FIG. 2A. However, the gaze angle θM is larger than the gaze angle θL or θR. The directions of the gaze to between the collimator plates 323 from the detection elements 322L, 322M, 322R differ. The difference in the gaze angle and the gaze direction leads to the difference in the quantity of scattered radiation incident on each detection element 322 without being absorbed by the collimator plates 323. Hence, the correction processing is performed using the correction function previously created for each of the detection elements 322L, 322M, 322R.

The creation of correction function is performed based on the process flow shown in FIG. 5 so that the correction function for the detection element 322M as well as those for the detection element 322L and the detection element 322R are created. The correction processing is performed according to the process flow shown in FIG. 7 so that the data acquired by the detection element 322M as well as the data acquired by the detection elements 322L and 322R are corrected.

### Second Embodiment

In the first embodiment, the description is made on the creation of the correction function for the detection element by using the direct radiation quantity and the scattered radiation quantity determined for each of the positions of plural detection elements 322 disposed between the collimator plates 323. In this embodiment, description is made on the creation of a correction function by using detection data acquired at each position of each detection element 322 in combination with detection data acquired at a position complementary to the relevant position. A schematic configuration of an X-ray CT apparatus of this embodiment is the same as that of the first embodiment and hence, the description thereof is dispensed with.

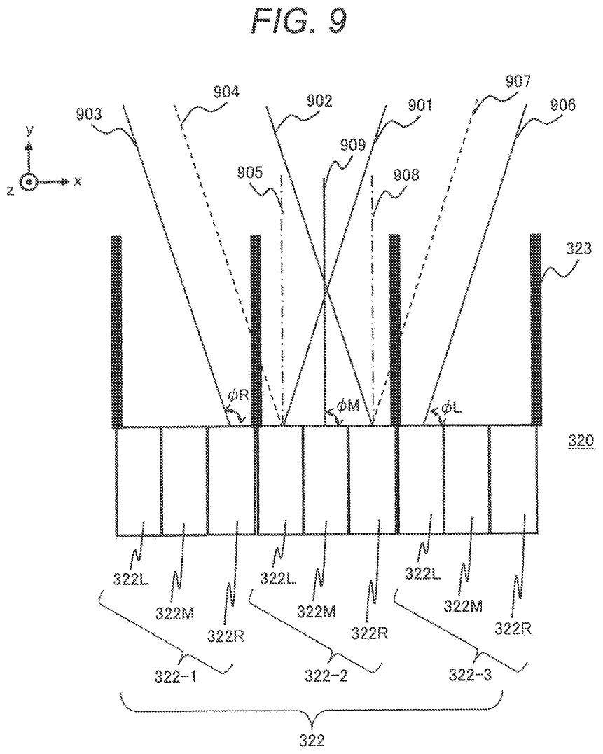

FIG. 9 is a fragmentary x-y sectional view of the X-ray detector 320, showing three groups 322-1, 322-2, 322-3 each of which includes three detection elements 322L, 322M and 322R between the collimator plates 323. As already described in the first embodiment, the direction φL of the gaze to between the collimator plates 323 from the detection element 322L and the direction φR of the gaze to between the collimator plates 323 from the detection element 322R are different and hence, the quantity of scattered radiation incident on the detection element 322L differs from that of scattered radiation incident on the detection element 322R. Further, the direction φM of the gaze to between the collimator plates 323 from the detection element 322M forms angles of 90° to the detection element and differs from the gaze directions φL and φR. Therefore, the quantity of scattered radiation incident on the detection element 322M differs from those of scattered radiation incident on the detection elements 322L and 322R.

In this embodiment, therefore, the difference of scattered radiation quantity is corrected by simulatively uniformizing the directions of the gaze to between the collimator plates 323 from the individual detection elements as the direction φM that forms angles of 90° to the element. Specifically, by taking advantage of the fact that the gaze directions φL and φR of the respective detection elements 322L and 322R are symmetrical, the detection data acquired by the detection element 322L is corrected with the detection data acquired by the detection element 322R.

In other words, the detection data acquired by the detection element 322L disposed at some position between the collimator plates 323 is corrected by using the detection data acquired by the detection element 322R disposed at a position complementary to the relevant position. The phase “complementary position” means that a direction of the gaze to between the collimator plates 323 from one detection element 322 is symmetrical to a direction of the gaze to between the collimator plates 323 from the other detection element 322″. For example, the detection element 322L and the detection element 322R are in a complementary positional relation while the detection element 322M disposed at the midpoint between the collimator plates 323 has no counterpart at the complementary position.

The gaze angle θM of the detection element 322M is larger than the gaze angle θL and θR of the detection elements 322L and 322R, as shown in FIG. 8. Hence, the quantity of scattered radiation incident on the detection element 322M is larger than the quantity of scattered radiation incident on the detection element 322L or the detection element 322R. It is therefore desirable to further perform correction by multiplication of gaze angle ratio.

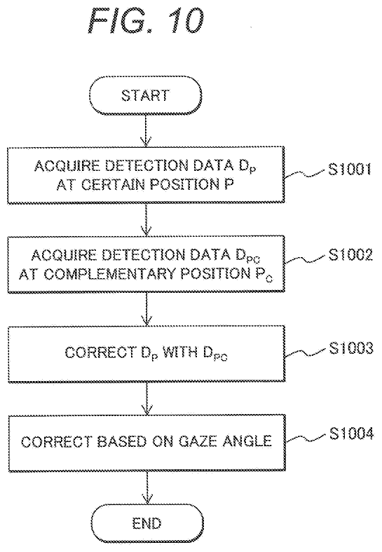

A process flow of the embodiment shown in FIG. 10 is described with reference to FIG. 8 and FIG. 9. The process flow of FIG. 10 is performed by the step S703 of FIG. 7.

### (S1001)

The data processor 420 acquires detection data D_{P} of the detection element 322 disposed at a certain position P between the collimator plates 323. For instance, detection data 901 of a detection element 322L in a detection element group 322-2 is acquired.

### (S1002)

The data processor 420 acquires detection data D_{Pc} of a detection element 322 disposed at a position Pc complementary to the position P. For instance, the data processor acquires detection data 902 of a detection element 322R in the detection element group 322-2 and in a complementary positional relation to the detection element 322L of the detection element group 322-2, and detection data 903 of a detection element 322R in the detection element group 322-1 and in the complementary positional relation to the detection element 322L of the detection element group 322-2. It is noted that plural detection elements 322 are disposed at positions Pc complementary to the position P. Although there are a plurality of detection elements 322 disposed at the position Pc commentary to the position P, for the purpose of computation reduction of the subsequent steps, it is preferred to select two detection elements 322 near the position P and to limitedly acquire the detection data of the two selected detection elements.

### (S1003)

The data processor 420 corrects the detection data D_{P} by using the detection data D_{Pc}. For instance, interpolation data 904 is calculated from the detection data 902 and the detection data 903. A mean value of the interpolation data 904 and the detection data 901 is calculated as correction data 905 for the detection element 322L of the detection element group 322-2. The interpolation data 904 is calculated by weighting addition based on a distance between the detection elements 322 and by using an equation {(detection data 902)+2×(detection data 903)}/3. The interpolation data is equivalent to detection data virtually acquired in the gaze direction φR of the detection element 322L of the detection element group 322-2.

The correction data 905 is a mean value of detection data pieces acquired by the detection element 322L of the detection element group 322-2 in the gaze directions φL and φR thereof. Hence, the gaze directions are uniformized as direction φM. The gaze directions are simulatively uniformized so that the difference in the scattered radiation quantity resulting from the different gaze directions is corrected. In FIG. 9, the solid line indicates the detection data, the dot line indicating the interpolation data, and the dot-dash line indicating the correction data.

The same processing may be performed to correct the detection data 902 of the detection element 322R of the detection element group 322-2 and to calculate correction data 908. Specifically, interpolation data 907 is calculated by using an equation {(detection data 901)+2×(detection data 906)}/3 and a mean value of the interpolation data 907 and the detection data 902 is calculated as the correction data 908. Namely, the correction processing in this step can be expressed as a correction function D_{P}cor using the following equation.

D_{P}cor=(D_{P}+D_{Pc}int)/2,

where D_{P} denotes detection data at a relevant position; D_{Pc}int={(n−m−1)×D_{Pc1}+(m+1)×D_{Pc2}}/n where n denotes the number of detection elements 322 between the collimator plates 323; m denotes the number of detection elements 322 between the closest complementary position and the relevant position; D_{Pc1} denotes detection data at the closest complementary position; and D_{Pc2} denotes detection data at the second closest complementary position. In a case where the closest complementary position adjoins the relevant position, m=0.

The detection element 322M disposed at the midpoint between the collimator plates 323 has the gaze direction φM. Hence, detection data 909 acquired by the detection element 322M of the detection element group 322-2, for example, does not require the correction of this step and is expressed as D_{Pc}int=0.

### (S1004)

The data processor 420 corrects the calculation result of the step S1003 based on the gaze angle. For instance, the difference of the scattered radiation quantity resulting from the different gaze angles is corrected by multiplying the calculated correction data 905 by a gaze angle ratio θM/θL.

By the above-described process flow, the detection data acquired at each of the positions of the plural detection elements 322 disposed between the collimator plates 323 is corrected by using the detection data acquired from the complementary position to the relevant position. Hence, the difference of the scattered radiation quantities caused by the different gaze directions of the respective detection elements 322 is corrected. Further, the difference of the scattered radiation quantities caused by the different gaze angles of the respective detection elements 322 is also corrected. Even in the case where the plural detection elements are disposed between the collimator plates, such correction processing can correct the difference of the scattered radiation quantities contained in the projection data so that the artifact on the tomographic image can be reduced.

The number of the detection elements 322 disposed between the collimator plates 323 is not limited to an odd number such as three but may be in an even number. In the case of an even number of detection elements 322 between the collimator plates as well, detection data acquired by a detection element 322 at a certain position P between the collimator plates 323 can be corrected by using detection data acquired by a detection element 322 disposed at a position Pc complementary to the relevant position P.

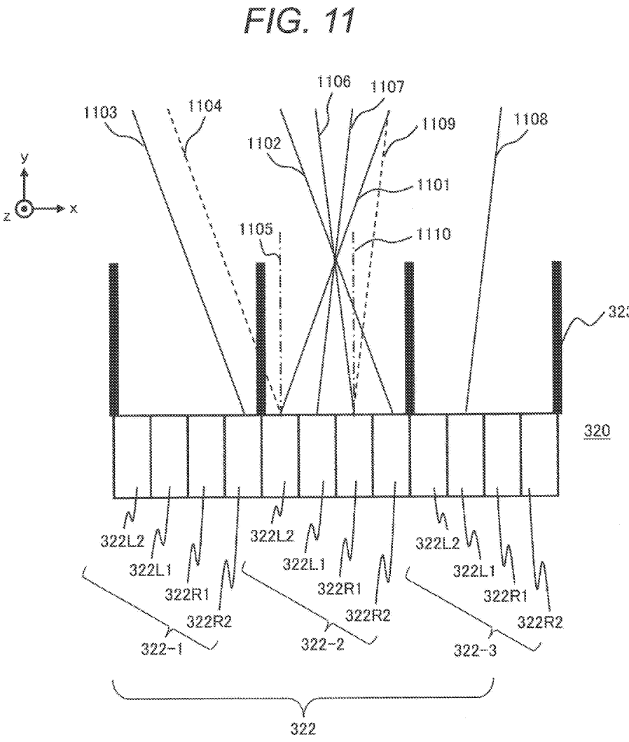

Now referring to FIG. 11, description is made on a case where four detection elements 322 are disposed between the collimator plates 323. FIG. 11 shows three groups 322-1, 322-2, 322-3 each of which includes four detection elements 322 between the collimator plates 323. In each group, a detection element 322L2, a detection element 322L1, a detection element 322R1, and a detection element 322R2 are arranged in this order from the left.

Now, description is made on steps of a procedure for correcting detection data 1101 of the detection element 322L2 of the detection element group 322-2. First, detection data 1102 of the detection element 322R2 in the detection element group 322-2 and in the complementary positional relation to the detection element 322L2 of the detection element group 322-2, and detection data 1103 of the detection element 322R2 of the detection element group 322-1 are acquired. The direction of the gaze to between the collimator plates 323 from the detection element 322L2 of the detection element group 322-2 is symmetrical to the gaze direction of the detection element 322R2 of the detection element group 322-2 and that of the detection element 322R2 of the detection element group 322-1. Next, interpolation data 1104 is calculated by weighting addition based on the distance between the detection elements 322 and by using the detection data 1102 and the detection data 1103. The detection element 322R2 of the detection element group 322-1 is the closest to and in complementary positional relation to the detection element 322L2 of the detection element group 322-2. Since these elements adjoin each other, m=0. Hence, the interpolation data is calculated using the equation (Interpolation data 1104)={(4−0−1)×(detection data 1103)+(0+1)×(detection data 1102)}/4. Then, a mean value of the interpolation data 1104 and the detection data 1101 is calculated as correction data 1105.

Similarly, description is made on steps of a procedure for correcting detection data 1106 of the detection element 322R1 of the detection element group 322-2. First, detection data 1107 of the detection element 322L1 in the detection element group 322-2 and in the complementary positional relation to the detection element 322R1 of the detection element group 322-2, and detection data 1108 of the detection element 322L1 of the detection element group 322-3 are acquired. Next, interpolation data 1109 is calculated by applying the detection data 1107 and the detection data 1108 in an equation {(4−0−1)×(detection data 1107)+(0+1)×(detection data 1108)}/4. Then, a mean value of the interpolation data 1109 and the detection data 1106 is calculated as correction data 1110.

Incidentally, the gaze angle of the detection element 322L1 and of the detection element 322R1 is larger than the gaze angle of the detection element 322L2 and of the detection element 322R2. It is therefore preferred to correct the correction data 1110 of the detection element 322R1 based on the gaze angle.

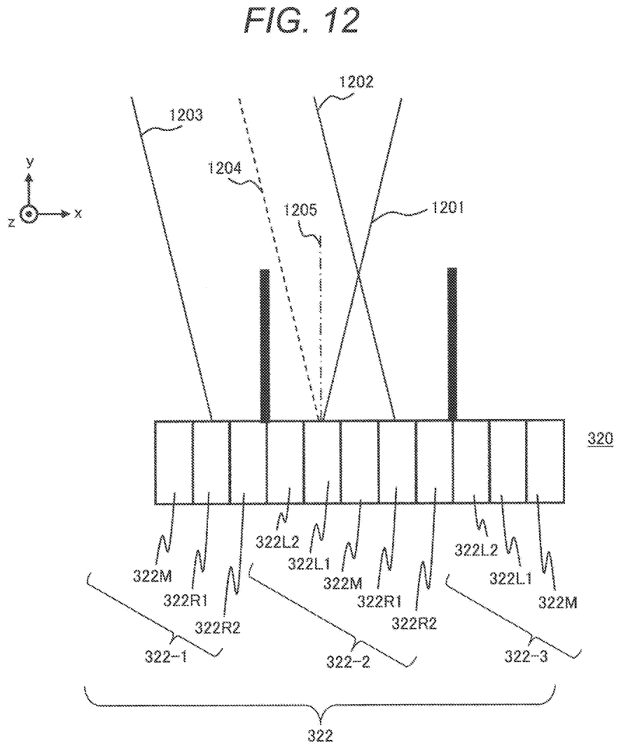

Now referring to FIG. 12, description is made on a case where five detection elements 322 are disposed between the collimator plates 323. FIG. 12 shows three groups 322-1, 322-2, 322-3 each of which includes five detection elements 322 between the collimator plates 323. In each group, the detection element 322L2, detection element 322L1, detection element 322M, detection element 322R1, and detection element 322R2 are arranged in this order from the left. In the detection element group 322-1, the detection element 322L2 and the detection element 322L1 are not shown. In the detection element group 322-3, the detection element 322R1 and the detection element 322R2 are not shown.

Now, description is made on steps of a procedure for correcting detection data 1201 of the detection element 322L1 of the detection element group 322-2. First, detection data 1202 of the detection element 322R1 in the detection element group 322-2 and in a complementary positional relation to the detection element 322L1 of the detection element group 322-2, and detection data 1203 of the detection element 322R1 of the detection element group 322-1 are acquired. The direction of the gaze to between the collimator plates 323 from the detection element 322L1 of the detection element group 322-2 is symmetrical to the gaze direction of the detection element 322R1 of the detection element group 322-2 and that of the detection element 322R1 of the detection element group 322-1. Next, interpolation data 1204 is calculated by weighting addition based on the distance between the detection elements 322 and using the detection data 1202 and the detection data 1203. The detection element 322R1 of the detection element group 322-2 is the closest to and in the complementary positional relation to the detection element 322L1 of the detection element group 322-2 and hence, m=1. Therefore, the interpolation data is calculated using the equation (Interpolation data 1204)={(5−1−1)×(detection data 1202)+(1+1)×(detection data 1203)}/5. Then, a mean value of the interpolation data 1204 and the detection data 1201 is calculated as correction data 1205. Correction data for the other detection elements 322 is calculated in the same way.

The plural embodiments of the present invention have been described as above. The present invention is not limited to the above embodiments, and the components thereof may be changed or modified without departing from the spirit and scope of the present invention. Some of the components disclosed herein may be combined as needed. Further, some of all the components illustrated by the above embodiments may be omitted.

100: X-ray CT apparatus, 110: object, 200: input/output section, 211: mouse, 212: keyboard, 213: monitor, 300: scanning section, 310: X-ray generator, 311: X-ray tube, 312: X-ray irradiation width controller, 320: X-ray detector, 322: detection element, 323: collimator plate, 330: gantry, 331: aperture, 332: rotating plate, 333: rotation driver, 340: scanning control unit, 341: X-ray controller, 342: gantry controller, 343: detector controller, 345: table controller, 346: integrated controller, 350: table, 400: image generation section, 410: data acquisition portion, 420: data processor, 421: central processing unit, 422: memory, 423: HDD, 901: detection data, 902: detection data, 903: detection data, 904: interpolation data, 905: correction data, 906: detection data, 907: interpolation data, 908: correction data, 909: detection data, 1101: detection data, 1102: detection data, 1103: detection data, 1104: interpolation data, 1105: correction data, 1106: detection data, 1107: detection data, 1108: detection data, 1109: interpolation data, 1110: correction data, 1201: detection data, 1202: detection data, 1203: detection data, 1204: interpolation data, 1205: correction data.

What is claimed is:

1. An X-ray CT apparatus comprising:
an X-ray irradiation section for X-ray radiation;
an X-ray detector including a plurality of detection elements for detecting the X-ray;
a plurality of collimator plates disposed between the X-ray irradiation section and the X-ray detector so as to reduce scattered radiation;
a reconstruction portion for reconstructing a tomographic image by using projection data generated based on an output from the X-ray detector; and
a correction portion for correcting the projection data by using different correction functions according to the positions of the plural detection elements disposed between the collimator plates.

2. The X-ray CT apparatus according to claim 1, wherein the correction function includes a quantity of direct radiation incident on the detection element and a quantity of scattered radiation incident on the detection element without being absorbed by the collimator plates, and the direct radiation quantity and the scattered radiation quantity are determined by Monte Carlo simulation.

3. The X-ray CT apparatus according to claim 2, wherein the correction function is created by fitting a ratio of the direct radiation quantity and the scattered radiation quantity by use of a quadratic function.

4. The X-ray CT apparatus according to claim 2,
wherein the correction function is stored in the form of a plurality of tables created for each object size and for each tube voltage, and
the correction portion retrieves a predetermined table from among the plural tables according to scanning conditions and uses the retrieved table for correction of the projection data.

5. The X-ray CT apparatus according to claim 1, wherein the correction function is used for correcting detection data acquired by a detection element at a certain position between the collimator plates by using detection data acquired by a detection element in complementary positional relation to the relevant position.

6. The X-ray CT apparatus according to claim 5, wherein the correction function is used for correcting the detection data at the relevant position by using detection data acquired by two detection elements at positions that are selected from among a plurality of complementary positions to the relevant position and are near the relevant position.

7. The X-ray CT apparatus according to claim 6, wherein the correction function is created by subjecting each of the detection data pieces acquired at the two selected positions to weighting addition based on a distance between each of the two selected positions and the relevant position.

8. The X-ray CT apparatus according to claim 5, wherein the correction function is used for correcting the detection data acquired by the detection element based on a gaze angle of the detection element between the collimator plates.

9. The X-ray CT apparatus according to claim 1, wherein the correction function is used for correcting the projection data based on a direction of the gaze to between the collimator plates from the detection element.

10. A correction method for correcting projection data generated by an X-ray CT apparatus including a plurality of detection elements disposed between a plurality of collimator plates, comprising:
a step of acquiring the projection data; and
a step of correcting the projection data by using different correction functions according to positions of the detection elements disposed between the collimator plates.
